(19) United States
(12) Patent Application Publication
(10) Pub. No.: US 2006/0129056 A1
(43) Pub. Date: Jun. 15, 2006

(54) Electrocorticography telemitter

(75) Inventors: Eric C. Leuthardt, St. Louis, MO (US); Daniel W. Moran, Ballwin, MO (US); Matthew P. LaConte, Maryland Hts., MO (US); Byron Kam, Kansas City, MO (US); Alexander Chieu, Charlotte, NC (US); Ralph G. Dacey, Ladue, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 11/011,018

(22) Filed: Dec. 10, 2004

(51) Int. Cl. A61B 5/04

(52) U.S. Cl. 600/544; 128/903

(57) ABSTRACT

Methods, systems, and articles of manufacture provide for wireless communications of brain signals from electrode implants to an external receiver for analysis of the brain signals. The analyzed brain signals are used to locate abnormal brain activity in a subject, such as epileptic seizure foci, or to localize task-specific brain activity.

### CROSS-REFERENCE TO RELATED APPLICATIONS

This application claims the benefit of prior filed U.S. patent application Ser. No. 10/734,370, filed Dec. 12, 2003, and the US provisional application converted therefrom, filed Dec. 10, 2004, the specifications of which are herein incorporated by reference in their entireties.

### STATEMENT REGARDING FEDERALLY SPONSORED RESEARCH OR DEVELOPMENT

Not Applicable.

### REFERENCE TO A SEQUENCE LISTING

Not Applicable.

### BACKGROUND OF THE INVENTION

1. Field of the Invention

The present invention relates in general to medical data sensing and communications devices, and more particularly to a wireless data transmission device and related methods for transmitting brain signals to a data acquisition and analysis device.

2. Description of the Related Art

Currently about two million people and about 50 million people worldwide suffer from epilepsy. One treatment approach is to eliminate seizures by surgical removal of brain tissue that is the focus of seizure activity. To do so, physicians map the cortex of a patient's brain by using a grid of sensors placed under the dura mater of the brain and recording brain activity for a period of up to two weeks. During this period, it is expected that the patient will experience a seizure, and data from the sensor array will provide information on the location of the seizure's origin in the brain. Neurosurgeons then use this information to evaluate the feasibility of removing brain tissue in the identified location in an effort to prevent seizure initiation.

However, current procedures for identifying the focus of seizure activity in a patient are arguably as risky, if not more so, than experiencing the seizures. Typically, a subdural electrode array is implanted beneath the protective dura mater covering the brain. Wire leads are attached to the array, and the leads tunnel out from under and through openings in the dura mater, the skull, and the scalp. The leads are coupled to a monitor and data storage device outside of the patient's body. This configuration provides a direct route of infection or invasion from the environment to the surface of the patient's brain, bypassing the body's normal protective barriers. The spinal fluid that bathes the brain and normally is contained by the dura mater leaks through the openings leading from the brain surface to the external environment.

Understandably then, patients undergoing this procedure experience very high rates of infection and other complications associated with this abnormal exposure of the brain. As many as 5% of patients undergoing the monitoring procedure suffer bacterial infection sufficiently serious to typically require an intense regimen of antibiotic therapy, intensive care and additional surgical procedures. The cost of the required added treatments in such cases can run into many tens of thousands of dollars.

Aside from the risks of infection, a patient undergoing the monitoring procedure at the very least experiences two weeks or so of severe discomfort. The electrode leads tether the patient to the computer monitoring equipment so that the patient's mobility is severely impaired, typically only permitting movement between a laying and a sitting position in bed. This severe limitation of activity places the patients at increased risk for complications related to prolonged bed rest which include deep venous thrombosis, pulmonary embolus, pneumonia, and bed sores. These problems are serious and potentially life-threatening, and increase the risks associated with invasive monitoring.

However, for patients with intractable epilepsy for whom surgery is the primary or perhaps the only viable treatment option, localization of the epileptic foci is an important prerequisite for complete evaluation of the surgical option. The localization procedure provides the surgeon with important information not only on the position of the seizure focus, but also on the extent of affected tissue so that no more tissue than is necessary is removed. A need therefore remains for improved systems and methods for locating the origins of epileptic seizures, and more generally for improved systems and methods of transmitting brain signals from implanted electrodes.

### BRIEF SUMMARY OF THE INVENTION

Methods, systems and articles of manufacture consistent with the present invention provide localization of seizure origin in a subject suffering from epilepsy. A wireless telemitter communicates brain signals such as electrocorticographic signals from subdural electrodes to a data acquisition and storage device. The brain signals are stored, and the location of brain signals and other characteristics of the brain signals are extracted. The location and other characteristics are analyzed to provide physicians with needed information for determining surgical procedure in the subject.

In accordance with articles of manufacture consistent with the present invention, a wireless telemitter includes multiple parallel modules each comprising a sensor array, each sensor array comprising multiple sensor electrodes for sensing the brain signals of a subject, and a plurality of sensor outputs, electronic circuitry coupled to the parallel modules for processing the brain signals from the sensor outputs, a wireless transmitter coupled to an output of the electronic circuitry, configured to transmit the processed brain signals from the sensor electrodes to a receiver external to the subject, and an inductively rechargeable battery for providing power to each module, wherein the wireless telemitter is fully implantable beneath the scalp of a subject.

In accordance with systems consistent with the present invention, a telemetry system for receiving and processing brain signals from a subject includes a fully implantable wireless telemitter including multiple identical parallel modules each including a sensor array, each sensor array including multiple sensor electrodes and sensor outputs and an inductively rechargeable battery for powering each module, and with respect to the subject, an external charging device for inductively recharging the battery through the skin of the subject.

In accordance with methods consistent with the present invention, a method for monitoring brain signals of a human subject includes implanting a wireless telemitter including sensor electrodes beneath the scalp of the subject, wherein the wireless telemitter is capable of sensing the brain signals of the subject and is further capable of wirelessly transmitting the brain signals to a signal receiving device, obtaining the brain signals of the subject with the wireless telemitter, and over a period of time, receiving with the signal receiving device the transmitted brain signals of the subject from the wireless telemitter.

Further in accordance with methods consistent with the present invention, a method for obtaining brain signals from a subject includes providing a fully implantable telemitter including multiple sensor electrodes coupled to a transmitter capable of wireless signal transmission, the transmitter being coupled to an inductively chargeable battery for powering the transmitter, and implanting the telemitter beneath the scalp of the subject.

Further in accordance with systems consistent with the present invention, a wireless transmission system for transmitting brain signals of a subject to an external receiving device includes multiple implantable sensing means for sensing the brain signals of the subject, implantable means for wireless transmission of the brain signals to the external receiving device, means for processing the brain signals of the subject, coupled to the brain signal sensing means, so that the brain signals are capable of providing input to the wireless transmission means, and implantable means for powering the brain signal sensing means and wireless transmission means, the powering means capable of being inductively recharged through the skin of the subject.

Other systems, methods, features, and advantages of the invention will become apparent to one with skill in the art upon examination of the following figures and detailed description. It is intended that all such additional systems, methods, features, and advantages be included within this description, be within the scope of the invention, and be protected by the accompanying drawings.

### BRIEF DESCRIPTION OF THE SEVERAL VIEWS OF THE DRAWINGS

The accompanying drawings, which are incorporated in and constitute a part of this specification, illustrate an implementation of the invention and, together with the description, serve to explain the advantages and principles of the invention. In the drawings,

FIG. 1a is a schematic diagram of a wireless subdural electrocorticography telemitter in accordance with an exemplary embodiment of the invention;

FIG. 1b is a schematic wiring diagram of the wireless subdural electrocorticography telemitter shown in FIG. 1a;

FIG. 2 is a schematic diagram of an electrocorticography sensor grid in accordance with an exemplary embodiment of the invention;

FIG. 3 is a schematic diagram of a shielded chip with built-in antenna structure for use in an electrocorticography telemitter;

FIG. 4 is a pin-diagram for the chip shown in FIG. 2;

FIG. 5 is a block diagram of signal processing in an electrocorticography telemitter;

FIG. 6 is a timing diagram for an exemplary multiplex (mux) in an electrocorticography telemitter;

FIG. 7 is a block diagram of a single channel of an exemplary amplifier used in an electrocorticography telemitter;

FIG. 8 is a graphical illustration of the frequency response of an exemplary low-pass filter (LPF) in the electrocorticography telemitter;

FIG. 9 is a schematic drawing showing the serial timing of an exemplary analog-to-digital converter in an electrocorticography telemitter; and

FIG. 10 is a circuit diagram of an implantable battery charging system in accordance with an electrocorticography telemitter.

### DETAILED DESCRIPTION OF THE INVENTION

Reference will now be made in detail to an implementation consistent with the present invention as illustrated in the accompanying drawings. Wherever possible, the same reference numbers will be used throughout the drawings and the following description to refer to the same or like parts.

Definitions

To facilitate understanding of the invention, certain terms as used herein are defined below as follows:

As used interchangeably herein, the terms “ECoG” and “electrocorticography” refer to the technique of recording the electrical activity of the cerebral cortex by means of electrodes placed directly on it, either under the dura mater (subdural) or over the dura mater (epidural) but beneath the skull.

ECoG activity recorded from the brain surface provides physicians with data that, among other things, localizes the foci of epileptic seizures. The ECoG signal is much more robust compared to electroencephalogram (EEG) signal: its magnitude is typically five times larger (0.05-1.0 mV versus 0.01-0.2 mV for EEG), its spatial resolution as it relates to electrode spacing is much finer (0.125 versus 3.0 cm for EEG), and its frequency bandwidth is more than double (0-100 Hz versus 0-40 Hz for EEG). ECoG signals represent a smaller population of neurons than does EEG, and the higher frequencies (gamma bands) in the bandwidth of ECoG are likely to be most closely associated with cortical function. Unlike EEG, ECoG is not contaminated by muscle electrical activity (EMG), and the skull improves the signal to noise ratio of the signal rather than attenuating the signal as with EEG. Since the ECoG signal is derived from subdural electrodes, the cortex does not need to be penetrated as with microelectrode systems. Therefore, scarring and subsequent encapsulation of the recording sites is less of a factor with ECoG electrodes than with intra-cortical microelectrodes. It is expected that these characteristics will translate to increased implant viability over time.

Methods, systems, and articles of manufacture consistent with the present invention provide communication of brain signals, such as ECoG signals, to a receiving device, such as a brain wave monitor or data acquisition hardware for collection and later analysis of the signals. Data characteristics the of collected brain signals are extracted by appropriately configured software, and the data characteristics then are used, for example, to locate the origin of abnormal brain activity, such as foci of epileptic seizures, or to localize task-specific brain activity.

As shown and described, the telemitter is characterized by the system metrics set forth in Table 1.

TABLE 1

| System Specifications | Metric |
|---|---|
| Distance of Transmission | About 5 cm-10 cm |
| Sampling Frequency | 100 Hz |
| Transmission Frequency | 1.4 GHz-1.7 GHz |
| Data | Continuous transmission of raw |
| | data for up to about 14 days |
| Sensor Range | 0.05-1 mV |
| Output | 50-1000 mV |
| Resolution | >12 bit |
| Thickness | <about 1 cm |

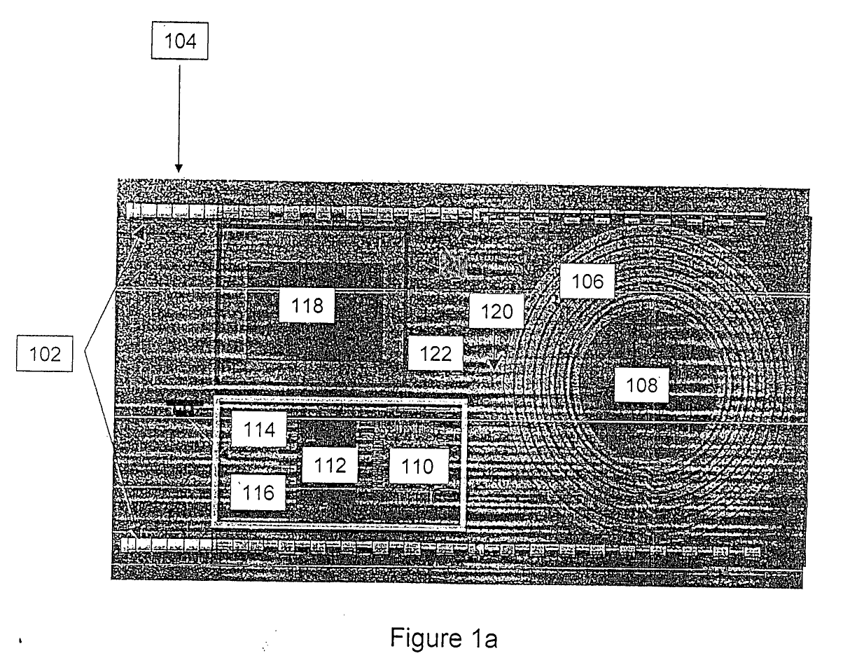

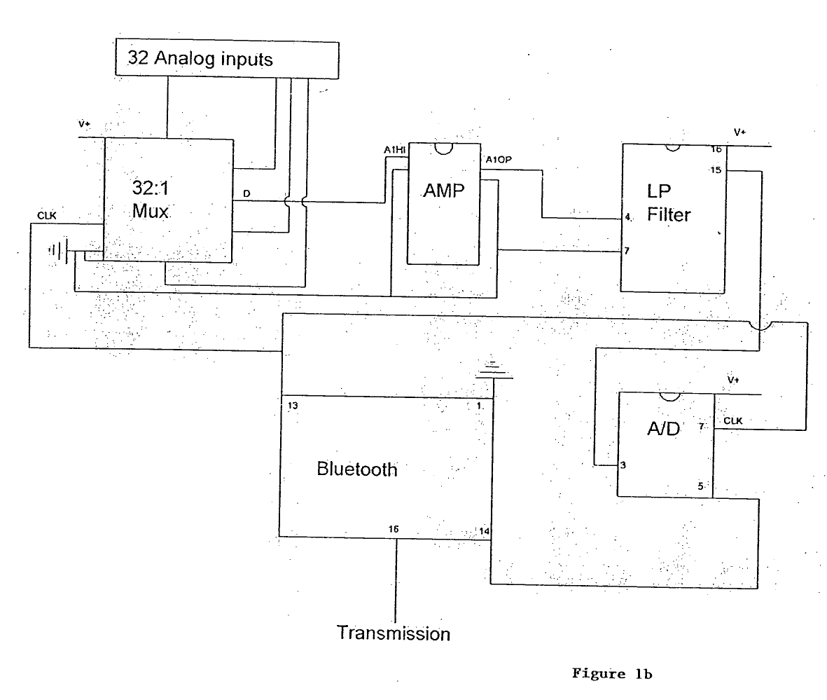

FIG. 1a is a schematic diagram of a wireless telemitter 100 consistent with the present invention. In an exemplary embodiment, telemitter 100 includes multiple input sensor array leads 102 for coupling with the signal outputs of multiple sensor electrode arrays. Further arranged on a flexible circuit board 104 are an inductive coil 106, a rechargeable lithium ion battery 108, a 32-1 multiplexing unit (Mux) 110, a +30 dB amplifier (Amp) 112, a low-pass filter 114, a 12-bit analog-to-digital (A/D) converter 116, a Bluetooth® wireless transmitter 118, and a recharging chip 120 for recharging battery 108. Circuit board 104 has a screw hole 122 to permit fixation of telemitter 100 to the skull. FIG. 1b is a schematic wiring diagram of telemitter 100.

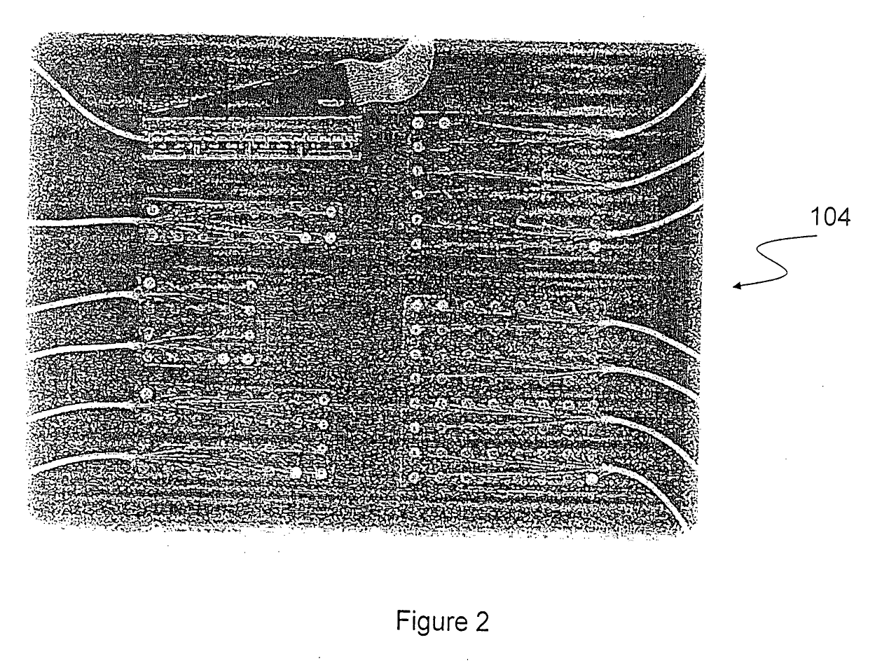

The sensor electrodes are, for example, subdural electrodes adapted for implantation beneath the dura mater. FIG. 2 shows a commercially available electrode sensor grid 104 that provides the sensor electrodes for an exemplary embodiment of telemitter 100. Sensor array leads from the sensor electrodes in sensor grid 104 slide into electrical contact with sensor array leads 102. Suitable subdural electrode arrays are those available from, for example, Ad-Tech Medical Instrument Corporation (Racine, Wis., USA). Such electrode grids are available in multiple sizes with varying numbers of channels, and telemitter 100 can accordingly be adapted to incorporate different sizes of grids and numbers of channel inputs, provided that the sizes of the grids are consistent with a multiple parallel module configuration as discussed below, that the remaining elements of telemitter 100 are adapted to synchronous management of the number of channels, and that the modules will fit within the confines of a closed scalp. In an exemplary embodiment, the modules are mounted on a flexible base so that the modules can be flexed to accommodate contours of the skull.

In an exemplary telemitter 100, four identical Ad-Tech Medical 32-sensor arrays are used in four identical, parallel modules to produce 128 channels of sensor input and output for telemitter 100. However, the modules need not be identical. The multiple parallel module arrangement provides both versatility and redundancy in signal monitoring. For example, with four modules of 32-channels each, a failure of any one module after implantation of the telemitter still leaves 96 sensors available for sensing and outputting the brain signals. Even so, replacement of any one failed module is a relatively more simple procedure than replacing an entire telemitter unit.

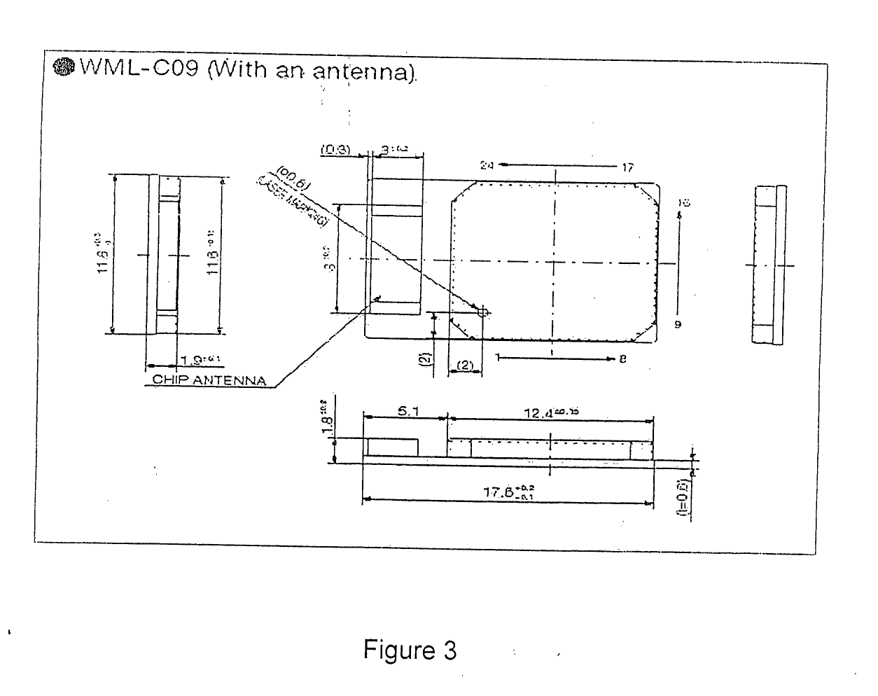

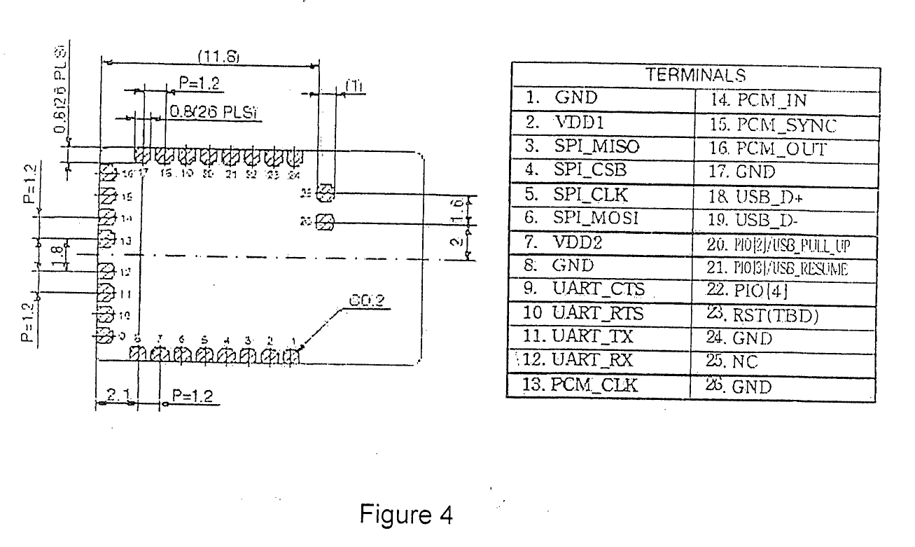

Wireless transmitter 118 is a Bluetooth® transmitter chip. FIG. 3 is a schematic diagram of a shielded Bluetooth chip with built-in antenna structure for use in telemitter 100. Telemitter 100 as shown uses the WML-C09 Class 2 Bluetooth Module, available from Mitsumi Electronics Corporation (headquartered in Tokyo, Japan, with head United States sales office in Dallas, Tex.). FIG. 4 is a pin-diagram for the WML-C09, which is characterized by a relatively compact size and relatively low power consumption, so that power from rechargeable battery 108 will be adequate to meet the needs of the chip. The WML-C09 dimensions are 11.8 mm (width) by 17.6 mm (length) by 1.9 mm (depth) and contributes to the flexibility of the exemplary telemitter 100. However, the precise size is not critical provided that the chip fits within the confines of the closed scalp when combined along with the other elements of the telemitter, and does not unduly interfere with the overall flexibility of the telemitter. The power consumption of the WML-C09 is relatively low, at 60 mA. Other Bluetooth wireless transmitter chips can be used, subject to the limitation that, since transmitter 118 is the largest power consumer in telemitter 100, the power specifications will be largely dictated by the power requirements of the selected chip and will be balanced against the size limitations on the battery.

The WML-C09 is further characterized as follows: 4 dB-output level, 721 kbps-transmission rate, Bluetooth Class 2 specification, Bluetooth v1.1 specification, high operational temperature range, shielded encasement, and built-in antenna. In addition, the internal logic of the WML-C09 provides for processing of the input signal into a wide variety of output formats. The internal processor automatically interfaces to UART, USB and PCM interfaces, which makes it adaptable to use with a wide range of receivers.

TABLE 2
WML-C09 Specifications:

| WML-C09 Specifications | Metric |
|---|---|
| Operational Frequency | 2402˜2480 MHz |
| Modulation | FHSS/GFSK |
| Channel Intervals | 1 MHz |
| Number of channels | 79 CH |
| Power Supply Voltage | 3.3 V |
| Current Consumption | 60 mA(typ) |
| Transmission Rate | 721 kbps |
| Receive Sensitivity | −80 dBm max. |
| Output Level (Class 2) | 4 dBm max. |
| Dimensions w/Antenna | 11.8(W) × 17.6(L) × 1.9(H) mm |

Bluetooth data transmission permits a single receiver to manage up to seven (7) implanted blue tooth transmitters asynchronously with low loss and low interference. The Gaussian pulse modulation tends to keep the transmission viable despite other RF interference (such as from inductive coil 106) by decoupling the data wave from both frequency modulated and amplitude modulated modes. Bluetooth Link Manager Protocol (LMP) establishes a clock-synchronized baseband of operation between one master (a receiver) and up to seven (7) slaves, then synchronously hops the whole “piconet” among 79 frequencies to minimize packet collisions with other piconets and to reduce interference from other devices operating in the same frequency band.

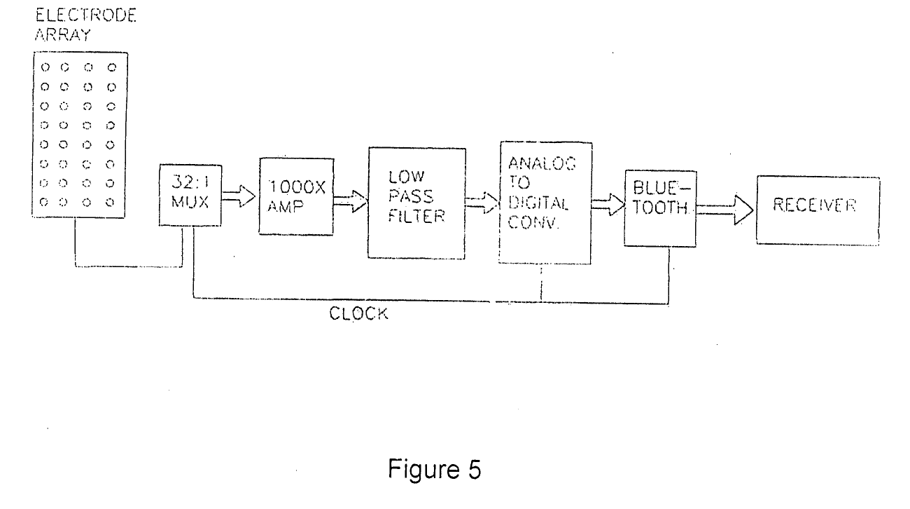

FIG. 5 is a block diagram of signal processing in telemitter 100, which describes the signal manipulation required to convert the analog signal produced by the sensor electrodes to a signal accepted by wireless transmitter 118. Generally, the signal is multiplexed, amplified, low-pass filtered and then converted from analog to digital.

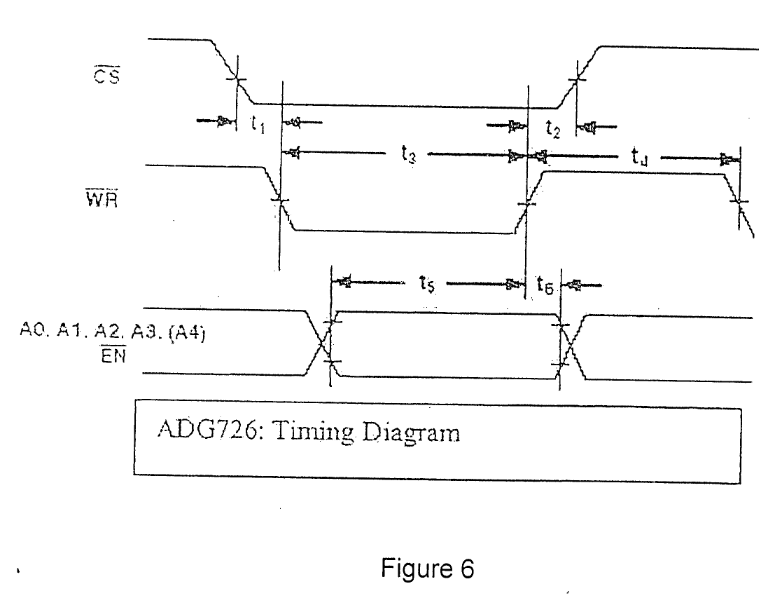

Thirty-two channels of analog data are input into the device at a sampling frequency of 250 Hz. To conserve power and maintain system simplicity, the 32 channels are multiplexed into a single channel at a frequency of 8 kHz. FIG. 6 is a timing diagram for an exemplary multiplexing unit (MUX) 110 in electrocorticography telemitter 100. In telemitter 100 as shown, the MUX is an ADG726 chip commercially available from Analog Devices (Norwood, Massachusetts). The MUX works by switching through all channels and holding the received channel for a length of time (t₆). The MUX holds a signal from a single channel by multiplying that channel by a constant voltage pulse. The pulse is then equivalent to the input signal during the hold time (t₆). During the transition time (t₄), the MUX switches to the next channel in line to add into the MUX output channel. Further details of the ADG726 are available from Analog Devices (see Analog Devices catalog or web site).

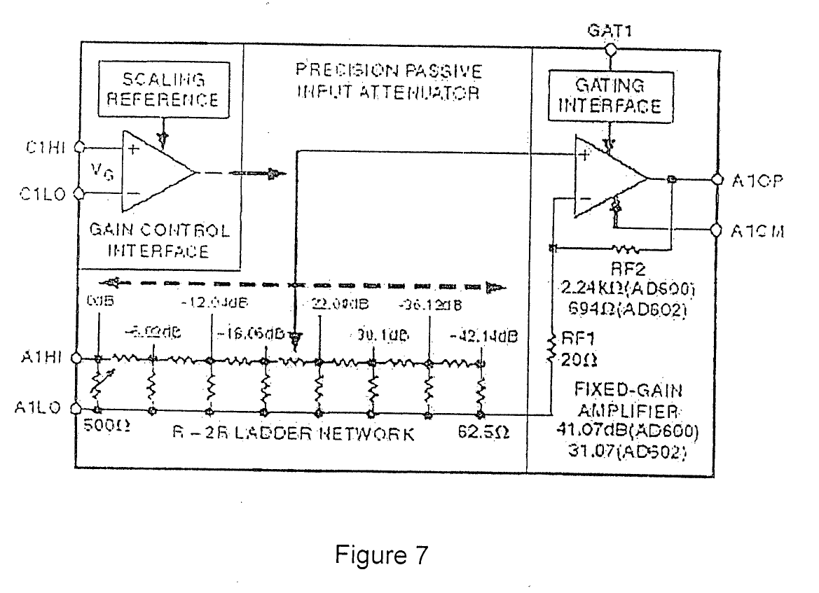

The voltage range of the ECoG signal from the sensor electrodes is approximately 0.05 mV-1.0 mV, which must be amplified for input into the A/D converter and to maintain resolution. In exemplary telemitter 100 as shown and described, the signal requires a +30 dB gain. FIG. 7 is a block diagram of a single channel of an exemplary amplifier 112 used in telemitter 100. The +30 dB Amp 112 is a 16-lead SOIC package (R-16) commercially available from Analog Devices.

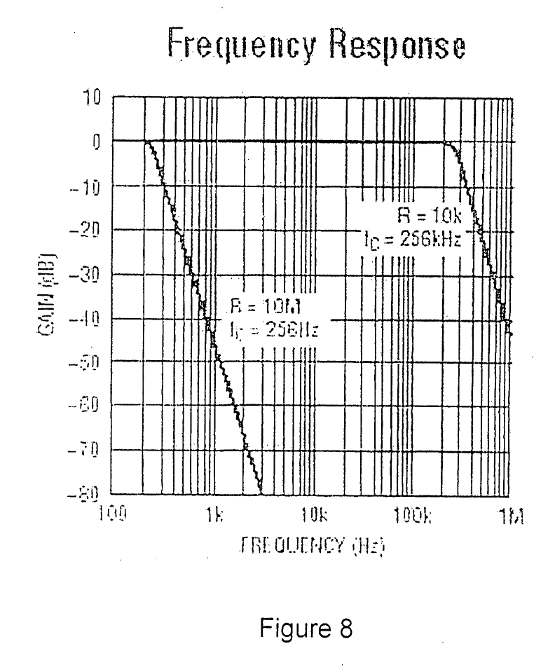

Low pass filtering by filter 114 is required of the amplified signal to remove high frequency distortions and prevent aliasing before the signal enters A/D converter 116. FIG. 8 is a graphical illustration of the frequency response of an exemplary low-pass filter (LPF) 114 used in telemitter 100 with a cutoff frequency of 105 Hz. As shown and described, telemitter filter 114 is a 28-lead plastic SSOP low-pass filter manufactured by Linear Technology and commercially available from Analog Devices.

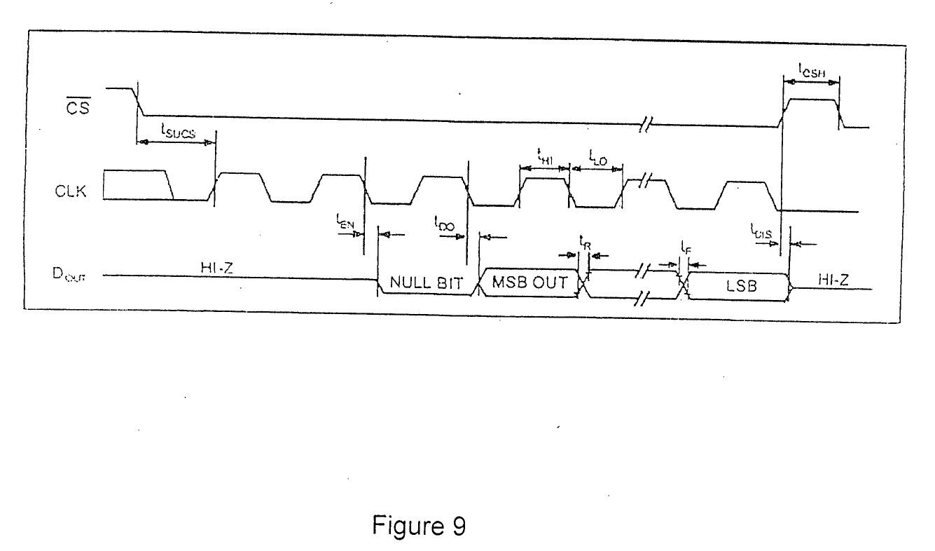

A/D converter 116 first synchronizes with an external clock provided by transmitter 118 and also synchronizes with MUX 110, and then sends a null bit before sampling of the analog data stream to produce a digital signal. The null bit provides a means for addressing each channel so that physicians can determine which electrode originates each signal in the electrode grid array. The location of each electrode thus becomes an indicator of seizure origin. Without addressing of the channels, the location information would be lost. FIG. 9 is a schematic drawing showing the serial timing of an exemplary A/D converter 116 used in telemitter 100 as shown and described. A/D converter 116 is, for example, an 8-lead plastic small outline and narrow (150 mil) chip available from Microchip Corporation (Itasca, IL).

Battery 108 is a rechargeable lithium-ion battery, such as the CGL3032 available from Panasonic. The CGL3032 measures only 30 mm in diameter, and 3.2 mm in height. With a charge capacity of 130 mAh, the CGL3032 is capable of powering telemitter for approximately 1 hour and 48 minutes on a full charge. However, other suitably compact and rechargeable batteries will be known to those of skill in the art. It is contemplated that improvements in battery technology will produce a sufficiently compact yet sufficiently long-lasting battery for use in telemitter 100 which must fit within the confines of the closed skull or scalp. However, currently available and sufficiently compact batteries do not maintain a charge or provide continuous power for the approximately two-week monitoring period. Accordingly, a charging system is required to recharge battery 108 periodically during the monitoring period.

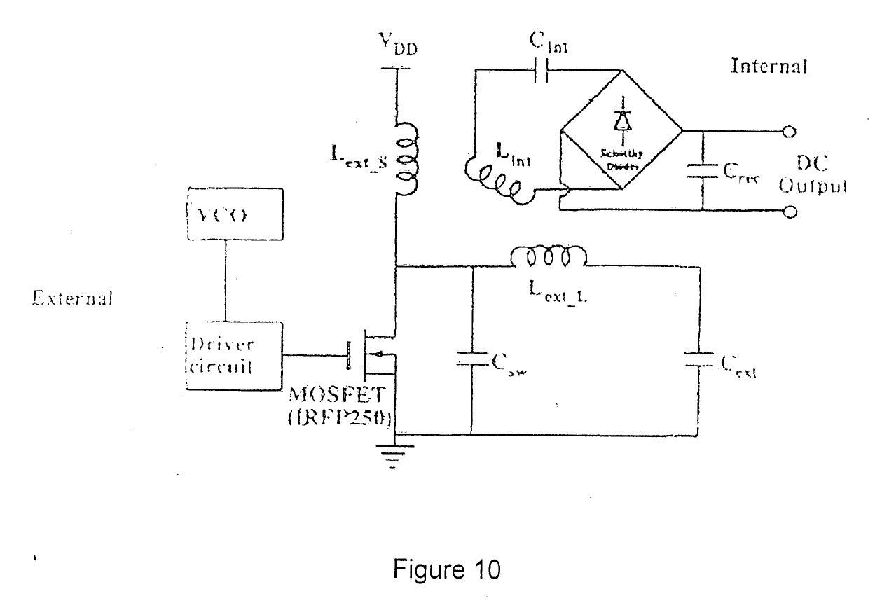

FIG. 10 is a circuit diagram of a charging system 120 for battery 108, and is a Transcutaneous Energy Transfer (TET) system consisting in part of inductive coils external to the subject's body, through which time-varying currents run to induce a current in inductive coil 106 in telemitter 100, which is implanted in the subject. The resulting current in inductive coil 106 charges battery 108 and powers telemitter 100.

The Panasonic CGL3032 requires a maximum constant charge current of 65 mA at 4.2 volts, and to be quickly charged, the charge current should remain as close to 65 mA as possible. As shown in FIG. 10, the TET system consists of separate, isolated external and internal systems. The external system consists of an oscillator that produces a sine wave output that is amplified. The external system also contains a MOSFET transistor able to tolerate high current and voltage. The amplified oscillating signal is then fed to two external inductor coils with capacitors that tune the circuit to resonant frequency, providing an efficient power transfer. The oscillator produces a range of radio frequency signals of 385-415 kHz, and an amplifier boosts the signal to get the voltage up to 9V and an AC current up to 2A. The time varying current flows through two external coils that induce current in internal coil 106. The TET system does not interfere with data transmission in Bluetooth transmitter 118, and the Bluetooth transmitter transmitting at a frequency of 2.4 GHz will not interfere with the TET system operating at frequencies of 385-415 kHz

As shown, telemitter 100 requires power of less than about 500 mW. To provide this power, the external system of the TET system contains capacitors to tune the circuit to the resonant frequency at which the circuit will operate most efficiently. An exemplary external system of the TET system uses a 22 nF inductor for the inductor with the larger outside diameter, and a 17 nF inductor for the smaller inductor. The external inductor takes the form of a coaxial dual-coil system, to maximize power efficiency and minimize misalignment of the inductors due to movement of the patient. The larger coil has 10 turns, with an outside diameter of 70 mm, and an inductance of 7.2 μH. The smaller coil has 13 turns, with an outside diameter of 40 mm, and an inductance of 9.2 μH. Internal coil 106 is configured to be as small as possible while maintaining the capability to induce sufficient current through telemitter 100. In the exemplary embodiment, internal coil 106 has 12 turns, an outside diameter of 38 mm, and an inductance of 4.1 μH. All inductor coils are fabricated from a type of wire consisting of multiple strands of individual wires that are insulated and braided together to form a single wire. Suitable wire is commercially available from Litz-Wire, Inc./HM Wire International, Inc.

In telemitter 100 as shown, using the Panasonic CGL3032 lithium ion battery, the charging circuit specifications require a charging current of less than 65 mA and a voltage of 4.2 volts. A charger chip that is capable of maintaining full control over the charging of the battery at required optimum levels is added to the charging circuit. A Maxim MAX745 switch-mode lithium ion battery charger chip, commercially available from Maxim Integrated Products, Inc. (Sunnyvale, Calif., and on the Web) is used in telemitter 100 as shown.

Flexible circuit board 104 provides a stable platform for the telemitter components, while at the same time providing sufficient flexibility for the telemitter to conform to the unique contours of a given patient's skull. A standard, rigid circuit board is not adaptable to implantation under the skull and adjacent to brain tissue. A suitable flexible circuit board is, for example, a multilayer board fabricated from a copper layer sandwiched between two outer layers of polyimide, available from All Flex, Inc. (Northfield, Minnesota). Other suitable flexible circuit boards will be apparent to those of skill in the art. In an alternative embodiment, wireless telemitter 100 includes the electronic components simply encased in a biocompatible casing such as the silicone casing described below, but without a flexible circuit board platform, with the components free-floating within the casing.

A casing of biocompatible material such as silicone encases telemitter 100. Suitable silicone material for fabricating the casing is available from, for example, Ad-Tech Medical. The casing is formed by pouring melted silicone into a mold of the shape telemitter 100. Once the silicone is cooled and partially congealed, telemitter 100 including circuitry and circuit board is placed on the silicone surface, and additional melted silicone is poured over the remaining exposed telemitter to fill the mold. Once the silicone and telemitter have cooled and the silicone casing is stable, the silicone-encased telemitter is removed from the mold, further cooled as necessary and can then be sterilized and sealed in a sterile package.

Further, the methods and systems consistent with the present invention provide benefits over conventional approaches, in that the telemitter is fully implantable beneath a closed scalp. More specifically, the telemitter provides a data transmission unit that is fully contained under a closed scalp after closure of the surgical incision for implanting the device. The telemitter is sterilizable and supplies physicians with up to about two weeks of sensor data in the form of ECoG signals of brain activity of the patient. The fully contained wireless telemitter will reduce the rate of infection to that of permanent implant procedures such as deep brain stimulators and cerebrospinal shunts. Additionally, by improving mobility and comfort, the risks associated with prolonged inactivity (i.e. pulmonary embolus, deep venous thrombosis, pneumonia, and bed sores) will be reduced.

The foregoing description of an implementation of the invention has been presented for purposes of illustration and description. It is not exhaustive and does not limit the invention to the precise form disclosed. Modifications and variations are possible in light of the above teachings or may be acquired from practicing the invention. The scope of the invention is defined by the claims and their equivalents.

What is claimed is:

1. A wireless telemitter comprising:
a plurality of parallel modules each comprising a sensor array, each sensor array comprising a plurality of sensor electrodes for sensing brain signals of a subject, and a plurality of sensor outputs;
electronic circuitry coupled to said plurality of parallel modules for processing the brain signals from said sensor outputs;
a wireless transmitter coupled to an output of said electronic circuitry, configured to transmit the processed brain signals from the sensor electrodes to a receiver external to the subject; and
an implantable power source without percutaneous wires for providing power to each module;
wherein the wireless telemitter is fully implantable beneath the scalp of a subject.

2. A wireless telemitter according to claim 1 wherein said implantable power source without percutaneous wires comprises an inductively rechargeable battery.

3. A wireless telemitter according to claim 2 wherein said inductively rechargeable battery is rechargeable by a Transcutaneous Energy Transfer (TET) system using magnetic induction.

4. A wireless telemitter according to claim 1 wherein said implantable power source without percutaneous wires comprises a battery.

5. A wireless telemitter according to claim 1 wherein said electronic circuitry for processing the brain signals from the sensor electrodes comprises:
a multiplexing unit coupled to said plurality of sensor outputs for multiplexing the brain signals from said plurality of sensor electrodes;
an amplifier coupled to said multiplexing unit for amplifying the multiplexed signal;
a low-pass filter coupled to said amplifier for filtering the amplified signal;
an analog-to-digital converter coupled to said low-pass filter for converting an analog signal from said sensor electrodes to a digital signal for said wireless transmitter.

6. A wireless telemitter according to claim 1 wherein said dedicated wireless transmitter comprises a Bluetooth module.

7. A wireless telemitter according to claim 6 wherein said Bluetooth module comprises a shielded chip having a built-in antenna.

8. A wireless telemitter according to claim 6 wherein said Bluetooth module comprises a WML-C09 Class 2 Bluetooth module.

9. A wireless telemitter according to claim 1 further comprising a casing comprising a biocompatible material encasing said plurality of parallel modules, said multiplexing unit, said amplifier, said low-pass filter, said analog-to-digital converter, said dedicated wireless transmitter, and said implantable power source without percutaneous wires.

10. A wireless telemitter according to claim 9 wherein said casing comprises silicone.

11. A wireless telemitter according to claim 1 further comprising a flexible circuit board for mounting said plurality of parallel modules, said multiplexing unit, said amplifier, said low-pass filter, said analog-to-digital converter, said dedicated wireless transmitter, and said implantable power source without percutaneous wires.

12. A wireless telemitter according to claim 11 further comprising a casing comprising a biocompatible material encasing said plurality of parallel modules, said multiplexing unit, said amplifier, said low-pass filter, said analog-to-digital converter, said dedicated wireless transmitter, said an implantable power source without percutaneous wires and said flexible circuit board.

13. A wireless telemitter according to claim 12 wherein said casing comprises silicone.

14. A telemetry system for receiving and processing brain signals from a subject, said telemetry system comprising:
a fully implantable wireless telemitter comprising a plurality of parallel modules each comprising a sensor array, each sensor array comprising a plurality of sensor electrodes and a plurality of sensor outputs, and an implantable power source without percutaneous wires for powering each module; and
with respect to the subject, an external charging device for inductively recharging said implantable power source through the skin of the subject.

15. A telemetry system according to claim 12 wherein said implantable power source without percutaneous wires comprises an inductively rechargeable battery that is rechargeable by a TET system using magnetic induction.

16. A telemetry system according to claim 15 wherein said wireless telemitter further comprises:
a multiplexing unit receiving a plurality of sensor outputs from said plurality of parallel modules;
an amplifier coupled to said multiplexing unit for amplifying the multiplexed signal;
a low-pass filter coupled to said amplifier for filtering the amplified signal;
an analog-to-digital converter coupled to said low-pass filter for converting an analog signal from said sensor electrodes to a digital signal; and
a dedicated wireless transmitter coupled to said analog-to-digital converter to receive the digital signal from said analog-to-digital converter and configured to transmit the signals from the sensor electrodes to an external receiver.

17. A wireless telemitter according to claim 16 wherein said dedicated wireless transmitter comprises a Bluetooth module.

18. A wireless telemitter according to claim 17 wherein said Bluetooth module comprises a shielded chip having a built-in antenna.

19. A wireless telemitter according to claim 18 wherein said Bluetooth module comprises a WML-C09 Class 2 Bluetooth module.

20. A wireless telemitter according to claim 16 further comprising a casing comprising a biocompatible material encasing said plurality of parallel modules, said multiplexing unit, said amplifier, said low-pass filter, said analog-to-digital converter, said dedicated wireless transmitter, and said implantable power source without percutaneous wires.

21. A wireless telemitter according to claim 20 wherein said casing comprises silicone.

22. A wireless telemitter according to claim 16 further comprising a flexible circuit board for mounting said plurality of parallel modules, said multiplexing unit, said amplifier, said low-pass filter, said analog-to-digital converter, said dedicated wireless transmitter, and said implantable power source without percutaneous wires.

23. A wireless telemitter according to claim 22 further comprising a casing comprising a biocompatible material encasing said plurality of parallel modules, said multiplexing unit, said amplifier, said low-pass filter, said analog-to-digital converter, said dedicated wireless transmitter, said implantable power source without percutaneous wires and said flexible circuit board.

24. A wireless telemitter according to claim 23 wherein said casing comprises silicone.

25. A method for monitoring brain signals of a human subject, said method comprising:
implanting a wireless telemitter including sensor electrodes beneath at least the scalp of the subject, wherein the wireless telemitter is capable of sensing the brain signals of the subject and is further capable of wirelessly transmitting the brain signals to a signal receiving device;
obtaining the brain signals of the subject with the wireless telemitter; and
over a period of time, receiving with the signal receiving device the transmitted brain signals of the subject from the wireless telemitter.

26. A method according to claim 25 further comprising providing the wireless telemitter, the wireless telemitter including a plurality of parallel modules each comprising a sensor array, each sensor array comprising a plurality of sensor electrodes and a plurality of sensor outputs;
a multiplexing unit coupled to said plurality of sensor outputs for multiplexing a plurality of signals from said plurality of sensor electrodes;
an amplifier coupled to said multiplexing unit for amplifying the multiplexed signal;
a low-pass filter coupled to said amplifier for filtering the amplified signal;
an analog-to-digital converter coupled to said low-pass filter for converting an analog signal from said sensor electrodes to a digital signal;
a dedicated wireless transmitter coupled to said analog-to-digital converter to receive the digital signal from said analog-to-digital converter and configured to transmit the signals from the sensor electrodes to an external receiver; and
an implantable power source without percutaneous wires for providing power to each module; and wherein the wireless telemitter is configured to be fully implantable within a subject's body.

27. A method according to claim 26 further comprising analyzing the received brain signals of the subject to locate an origin of abnormal brain activity within the brain of the subject.

28. A method according to claim 26 further comprising inductively recharging the battery at least once.

29. A method according to claim 25 wherein implanting the wireless telemitter beneath the scalp of the subject comprises implanting the wireless telemitter beneath the scalp and beneath the dura mater of the subject.

30. A method for obtaining brain signals from a subject, said method comprising:
providing a fully implantable telemitter including a plurality of sensor electrodes coupled to a transmitter capable of wireless signal transmission, the transmitter coupled to an implantable power source without percutaneous wires for powering the transmitter; and
implanting the telemitter beneath the scalp of the subject.

31. A method according to claim 30 wherein providing a telemitter further comprises:
providing a telemitter including:
a multiplexing unit coupled to the plurality of sensor electrodes outputs for multiplexing the signals from the plurality of sensor electrodes;
an amplifier coupled to the multiplexing unit for amplifying the multiplexed signal;
a low-pass filter coupled to the amplifier for filtering the amplified signal; and
an analog-to-digital converter receiving an output of the low-pass filter for converting an analog signal from the sensor electrodes to a digital signal for the wireless transmitter.

32. A wireless transmission system for transmitting brain signals of a subject to an external receiving device, said system comprising:
a plurality of implantable sensing means for sensing the brain signals of the subject;
implantable means for wireless transmission of the brain signals to the external receiving device;
means for processing the brain signals of the subject, coupled to the brain signal sensing means, so that the brain signals are capable of providing input to the wireless transmission means; and
implantable means for powering the brain signal sensing means and wireless transmission means, said powering means without percutaneous wires and capable of being implanted beneath the skin of the subject.

33. A wireless transmission system according to claim 32 wherein the signal processing means comprises:
multiplexing means coupled to the sensing means, for multiplexing a signals from the plurality of brain signal sensing means;
an amplifier coupled to the multiplexing means, for amplifying the multiplexed signal;
a means for low-pass filtering coupled to the amplifier, for filtering the amplified signal; and
means for converting an analog output of the low-pass filtering means to a digital output for the wireless transmitter.
